(19) United States
(12) Patent Application Publication
(10) Pub. No.: US 2025/0221641 A1
(43) Pub. Date: Jul. 10, 2025

(54) SYSTEMS, DEVICES, AND METHODS FOR PERFORMING TRANS-ABDOMINAL FETAL OXIMETRY AND/OR TRANS-ABDOMINAL FETAL PULSE OXIMETRY USING INDEPENDENT COMPONENT ANALYSIS

(71) Applicant: Raydiant Oximetry, Inc., San Ramon, CA (US)

(72) Inventors: Neil Padharia Ray, Sacramento, CA (US); Mark Andrew Rosen, Piedmont, CA (US); Adam Jacobs, Hollis, NH (US); Kenneth Holt, Cacadero, CA (US)

(21) Appl. No.: 19/017,755

(22) Filed: Jan. 12, 2025

Related U.S. Application Data
(63) Continuation of application No. 18/596,612, filed on Mar. 5, 2024, now Pat. No. 12,226,209.
Application No. 18/596,612 is a continuation of application No. 17/869,251, filed on Jul. 20, 2022, now Pat. No. 11,937,925.
Application No. 17/869,251 is a continuation of application No. 16/958,136, filed on Jun. 25, 2020, now Pat. No. 11,419,530.
(60) Provisional application No. 62/611,849, filed on Dec. 29, 2017.

(51) Int. Cl. A61B 5/1464; A61B 5/00; A61B 5/03; A61B 5/08; A61B 5/145; A61B 5/1455

(52) U.S. Cl. CPC A61B 5/1464; A61B 5/14546; A61B 5/14552; A61B 5/7203; A61B 5/7225; A61B 5/033; A61B 5/08; A61B 2562/0238; A61B 2562/046

(57) ABSTRACT

Independent component analysis may be performed on a plurality of detected electronic signals to separate signals within the detected electronic signals that are contributed by different sources. Each of the plurality of detected electronic signals may be received from a separate detector and may correspond to a detected optical signal emanating from a pregnant mammal's abdomen and a fetus contained therein. The detected optical signals may correspond to light that is projected into the pregnant mammal's abdomen from a light source. The separated signals may be analyzed to determine a separated signal that corresponds to light incident upon the fetus, which may be analyzed to determine a fetal hemoglobin oxygen saturation level of the fetus. An indication of the fetal hemoglobin oxygen saturation level may then be provided to the user.

### RELATED APPLICATION

This application is a Continuation of U.S. application Ser. No. 18/596,612 entitled “SYSTEMS, DEVICES, AND METHODS FOR PERFORMING TRANS-ABDOMINAL FETAL OXIMETRY AND/OR TRANS-ABDOMINAL FETAL PULSE OXIMETRY USING INDEPENDENT COMPONENT ANALYSIS” filed 5 Mar. 2024, which is a Continuation of U.S. application Ser. No. 17/869,251 entitled “SYSTEMS, DEVICES, AND METHODS FOR PERFORMING TRANS-ABDOMINAL FETAL OXIMETRY AND/OR TRANS-ABDOMINAL FETAL PULSE OXIMETRY USING INDEPENDENT COMPONENT ANALYSIS” filed 20 Jul. 2022, which is a Continuation of U.S. application Ser. No. 16/958,136 entitled “SYSTEMS, DEVICES, AND METHODS FOR PERFORMING TRANS-ABDOMINAL FETAL OXIMETRY AND/OR TRANS-ABDOMINAL FETAL PULSE OXIMETRY USING INDEPENDENT COMPONENT ANALYSIS” filed 25 Jun. 2020, which is a U.S. National Phase Application of PCT/US2018/068042 entitled “SYSTEMS, DEVICES, AND METHODS FOR PERFORMING TRANS-ABDOMINAL FETAL OXIMETRY AND/OR TRANS-ABDOMINAL FETAL PULSE OXIMETRY USING INDEPENDENT COMPONENT ANALYSIS” filed 28 Dec. 2018, which is a non-provisional of, and claims priority to, U.S. Provisional Patent Application No. 62/611,849 entitled “SYSTEMS, DEVICES, AND METHODS FOR PERFORMING TRANS-ABDOMINAL FETAL OXIMETRY AND/OR TRANS-ABDOMINAL FETAL PULSE OXIMETRY USING INDEPENDENT COMPONENT ANALYSIS” filed 29 Dec. 2017, all of which are incorporated by reference, in their entireties, herein.

### FIELD OF INVENTION

The present invention is in the field of medical devices and, more particularly, in the field of trans-abdominal fetal oximetry and trans-abdominal fetal pulse oximetry.

### BACKGROUND

Oximetry is a method for determining the oxygen saturation of hemoglobin in a mammal's blood. Typically, 90% (or higher) of an adult human's hemoglobin is saturated with (i.e., bonded to) oxygen while only 30-60% of a fetus's blood is saturated with oxygen. Pulse oximetry is a type of oximetry that uses changes in blood volume through a heartbeat cycle to internally calibrate hemoglobin oxygen saturation measurements of the arterial blood.

Current methods of monitoring fetal health, such as monitoring fetal heart rate, are inefficient at determining levels of fetal distress and, at times, provide false positive results indicating fetal distress that may result in the unnecessary performance of a Cesarean section.

### SUMMARY

Systems, devices, and methods for performing trans-abdominal fetal oximetry and/or trans-abdominal fetal pulse oximetry using independent component analysis are herein described. In one embodiment, a plurality of detected electronic signals may be received. Each of the plurality of detected electronic signals may be received from a separate detector (e.g., a photodetector) communicatively coupled to the processor and may correspond to a detected optical signal emanating from a pregnant mammal's abdomen and a fetus contained therein. Each detected optical signal may be converted, by the respective detector, into one of the plurality of the detected electronic signals. The detected optical signals may correspond to light that is projected into the pregnant mammal's abdomen from a light source. The light may include two or more different wavelengths, or ranges of wavelengths, of light. For example, the light projected into the pregnant mammal's abdomen may be within the range of red, near infrared, and/or infrared light.

Independent component analysis may then be performed on the plurality of detected electronic signals to separate signals within the detected electronic signals that are contributed by different sources (e.g., maternal photoplethysmogram, a fetal photoplethysmogram, a maternal respiratory signal, a uterine tone signal, and/or a noise signal). Each of the separated signals may correspond to a different source.

The separated signals may be analyzed to determine a separated signal that corresponds to light incident upon the fetus. In some embodiments, the separated signal that corresponds to light incident upon the fetus is a fetal photoplethysmogram signal. The separated signal that corresponds to light incident upon the fetus may be analyzed to determine a fetal hemoglobin oxygen saturation level of the fetus. An indication of the fetal hemoglobin oxygen saturation level may then be provided to the user. At times, the fetal oxygen saturation level may be provided to the user as a numerical value or as a value depicted on a graph. In some instances, the fetal hemoglobin oxygen saturation level may be provided as a time weighted average taken over, for example, 30 seconds, 1, 2, 5, 10, 20, and/or 30 minutes.

In some embodiments, a separated signal that corresponds to light incident upon the pregnant mammal (e.g., maternal photoplethysmogram, a maternal respiratory signal, a uterine tone signal, etc.) may be analyzed to determine, for example, a condition of the pregnant mammal and/or how the separated signal that corresponds to light incident upon the pregnant mammal contributes to the initially received detected electronic signals. Then, an indication of the analysis results may be provided to the user.

In some embodiments, the received plurality of detected electronic signals may be filtered and/or amplified prior to performance of the independent component analysis. At times, this filtering and/or amplification may incorporate use of a received secondary signal (e.g., maternal heart rate, fetal heart rate, noise, etc.). When a secondary signal is received, it may be synchronized in time and/or phase with the secondary signal prior to filtering the received plurality of detected electronic signals.

In another embodiment, a processor may receive a first detected electronic signal from a first detector, a second detected electronic signal from a second detector, and a third detected electronic signal from a third detector. Each of the first, second, and third detectors may be communicatively coupled to the processor. Each of the first, second, and third detected electronic signals may correspond to a detected optical signal emanating from a pregnant mammal's abdomen and a fetus contained that is detected by the respective first, second, and third detectors and converted into the respective first, second, and third detected electronic signals. The detected optical signal may correspond to an optical signal projected into the pregnant mammal's abdomen by one or more light sources.

The processer may perform independent component analysis on the first, second, and third detected electronic signals to generate a first separated signal, a second separated signal, and a third separated signal. The first separated signal, second separated signal, and third separated signal may be contributed to the first, second, and third detected electronic signals by a first source, a second source, and a third source, respectively. One of the separated signals (e.g., the third separated signal) may correspond to a fetal photoplethysmogram signal. The separated signal that corresponds to the fetal photoplethysmogram signal may then be analyzed to determine a hemoglobin oxygen saturation level of the fetus. Provision of an indication of the hemoglobin oxygen saturation level of the fetus to a user may then be facilitated.

In some embodiments, the first source of the first separated signal may be motion artifacts of the pregnant mammal, respiration of the pregnant mammal, photoplethysmogram variations of the pregnant mammal, uterine tone of the pregnant mammal, or noise.

In some embodiments, the second source of the second separated signal may be motion artifacts of the pregnant mammal, respiration of the pregnant mammal, photoplethysmogram variations of the pregnant mammal, uterine tone of the pregnant mammal, and noise. In most cases, the source of the second source will not be the same as the first source.

At times, a fourth detected electronic signal from a fourth detector may be received by the processor prior to performance of the independent component analysis. The fourth detected electronic signal may correspond to a detected optical signal emanating from the pregnant mammal's abdomen and contained fetus that is detected by the fourth detector and converted into the fourth detected electronic signal. The independent component analysis may be performed on/using the first, second, third, and fourth detected electronic signals to generate the first separated signal, the second separated signal, the third separated signal, and a fourth separated signal. The fourth separated signal may be contributed by a fourth source. Exemplary fourth sources include, but are not limited to, motion artifacts of the pregnant mammal, respiration of the pregnant mammal, photoplethysmogram variations of the pregnant mammal, uterine tone of the pregnant mammal, and noise.

### BRIEF DESCRIPTION OF THE FIGURES

The present invention is illustrated by way of example, and not limitation, in the figures of the accompanying drawings in which:

FIG. 1 is a block diagram illustrating an exemplary system for determining a fetal hemoglobin oxygen saturation level, consistent with some embodiments of the present invention;

FIG. 2A provides an illustration of an exemplary fetal hemoglobin probe with three detectors, consistent with some embodiments of the present invention;

FIG. 2B provides an illustration of an exemplary fetal hemoglobin probe with five detectors, consistent with some embodiments of the present invention;

FIG. 3 is a flowchart illustrating an exemplary process for performing fetal oximetry and/or fetal pulse oximetry trans-abdominally and/or in-utero to determine fetal hemoglobin oxygen saturation level using independent component analysis, consistent with some embodiments of the present invention;

FIG. 4 is a flowchart illustrating an exemplary process for performing fetal oximetry and/or fetal pulse oximetry trans-abdominally and/or in-utero to determine fetal hemoglobin oxygen saturation level using independent component analysis, consistent with some embodiments of the present invention;

FIG. 5 is a flowchart illustrating an exemplary process for performing fetal oximetry and/or fetal pulse oximetry trans-abdominally and/or in-utero to determine fetal hemoglobin oxygen saturation level using independent component analysis, consistent with some embodiments of the present invention; and

FIG. 6 depicts components of a computer system in which computer readable instructions instantiating the methods of the present invention may be stored and executed, consistent with some embodiments of the present invention.

### DESCRIPTION

Described herein are systems, devices, and methods for performing transabdominal fetal oximetry and/or fetal pulse oximetry. A key output of fetal oximetry and/or fetal pulse oximetry is the level of oxygen saturation of the fetus's blood (also referred to herein as “fetal hemoglobin oxygen saturation level” and “oxygen saturation level”) which may also be understood as the percentage of hemoglobin present in the fetus' blood that is bound to oxygen. The oxygen saturation level of a fetus' blood may be used by trained medical professionals to assess the health of a fetus (e.g., a level of hypoxia or fetal acidosis) as well as a level of stress it may be under during, for example, a labor and delivery process. Typically, values of oxygen saturation for fetal blood fall within the range of 30-60% with anything lower than 30% indicating that the fetus may be in distress.

For the purposes of the following discussion, the terms “pregnant mammal” or “maternal” or “mother” is used to refer to a female human being or animal (e.g., horse or cow) pregnant with a fetus. In most embodiments, the pregnant individual will be a human being, but this need not be the case as the invention may be used for nearly any pregnant mammal.

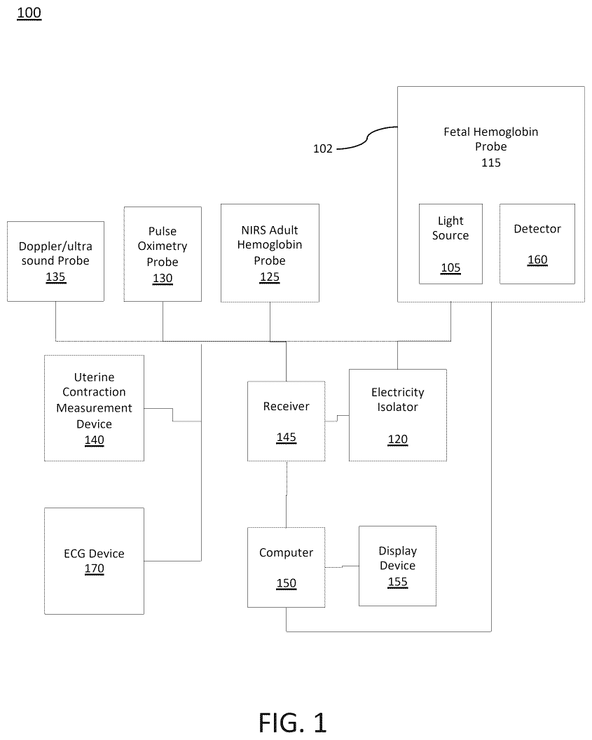

FIG. 1 is a block diagram illustrating an exemplary system 100 for performing fetal oximetry and/or fetal pulse oximetry trans-abdominally and/or in-utero to determine fetal hemoglobin oxygen saturation level using independent component analysis. The components of system 100 may be coupled together via wired and/or wireless communication links. In some instances, wireless communication of one or more components of system 100 may be enabled using short-range wireless communication protocols designed to communicate over relatively short distances (e.g., BLUETOOTH®, near field communication (NFC), radio-frequency identification (RFID), and Wi-Fi) with, for example, a computer or personal electronic device (e.g., tablet computer or smart phone) as described below. Electrical power may be supplied to system 100 and/or any component thereof via, for example, an on-board power supply (e.g., battery) and/or a coupling (e.g., plug) to an external power supply (e.g., electrical main). In some instances, a battery may be rechargeable.

System 100 includes a light source 105 and a detector 160 that, at times, may be housed in a single housing, which may be referred to as fetal hemoglobin probe 115. In some instances, fetal hemoglobin probe 115 may be a pulse oximetry device or pulse oximeter. Light source 105 may include a single, or multiple light sources and detector 160 may include a single, or multiple detectors.

Light source(s) 105 may transmit light at light of one or more wavelengths, including light within the red, near infra-red (NIR), and/or infrared bands of the electromagnetic spectrum into the pregnant mammal's abdomen. Typically, the light emitted by light source(s) 105 will be focused or emitted as a narrow beam to reduce spreading of the light upon entry into the pregnant mammal's abdomen.

Light source(s) 105 may be, for example, a LED, and/or a LASER, a tunable light bulb and/or a tunable LED that may, in some instances, be coupled to a fiber optic cable. In some instances, the light source(s) 105 may be tunable or otherwise user configurable while, in other instances, one or more of the light sources may be configured to emit light within a pre-defined range of wavelengths. Additionally, or alternatively, one or more filters (not shown) and/or polarizers may filter/polarize the light emitted by light source(s) 105 to be of one or more preferred wavelengths. These filters/polarizers may also be tunable or user configurable.

In some embodiments, light source(s) 105 may be an array of two or more light source(s) 105. An exemplary light source 105 is one with a relatively small form factor and high efficiency so as to limit heat emitted by the light source 105. In one embodiment, light source 105 is configured to emit light at 850 nm an example of which is the LED in Dragon Dome Package that Emits Light of 850 nm manufactured by OSRAM Opto Semiconductors (model number SFH 4783), which has a length of 7.080 mm and a width of 6.080 mm.

In some embodiments, one or more light sources 105 may be a fiber optic cable transmitting light produced by another source (e.g., a LASER or tunable light bulb or LED) not resident within fetal hemoglobin probe 115. In some instances, the light source(s) 105 may be tunable or otherwise user configurable while, in other instances, one or more of the light sources may be configured to emit light within a pre-defined range of wavelengths. Additionally, or alternatively, one or more filters (not shown) and/or polarizers may filter/polarize the light emitted by light source(s) 105 to be of one or more preferred wavelengths. These filters/polarizers may also be tunable or user configurable.

An exemplary light source 105 may have a relatively small form factor and may operate with high efficiency, which may serve to, for example, conserve space and/or limit heat emitted by the light source 105. In one embodiment, light source 105 is configured to emit light in the range of 770-850 nm. Exemplary flux ratios for light source(s) include but are not limited to a luminous flux/radiant flux of 175-260 mW, a total radiant flux of 300-550 mW and a power rating of 0.6 W-3.5 W.

Detector 160 may be configured to detect a light signal emitted from the pregnant mammal and/or the fetus via, for example, transmission and/or back scattering. Detector 160 may convert this light signal into an electronic signal, which may be communicated to a computer or processor and/or an on-board transceiver that may be capable of communicating the signal to the computer/processor. The optical light detected and converted into an electrical signal by detector 160 may be referred to herein as a detected electronic signal. This detected electronic signal might then be processed in order to determine how much light, at various wavelengths, is incident upon, scattered by, and/or absorbed by the fetal oxyhemoglobin and/or de-oxyhemoglobin so that a fetal hemoglobin oxygen saturation level may be determined. This processing will be discussed in greater detail below.

Exemplary detectors 160 include, but are not limited to, cameras, traditional photomultiplier tubes (PMTs), silicon PMTs, avalanche photodiodes, and silicon photodiodes. In some embodiments, the detectors may have a relatively low cost (e.g., $50 or below), a low voltage requirement (e.g., less than 100 volts), and non-glass (e.g., plastic) form factor. However, these alternatives do not have the same sensitivity as PMTs. In other embodiments, (e.g., contactless pulse oximetry) a sensitive camera may be deployed to receive light exiting the pregnant mammal's abdomen. For example, detector 160 may be a sensitive camera adapted to capture small changes in fetal skin tone caused by changes in cardiovascular pressure as the fetus' heart beats. In these embodiments, detector 160 and/or fetal hemoglobin probe 115 may be in contact with the pregnant mammal's abdomen, or not, as this embodiment may be used to perform so-called contactless pulse oximetry. In these embodiments, light source(s) 105 may be adapted to provide light (e.g., in the visible spectrum, near-infrared, etc.) directed toward the pregnant mammal's abdomen so that the detector 160 is able to receive/detect light exiting the pregnant mammal's abdomen and fetus. The light captured by detector 160 may be communicated to computer 150 for processing to convert the images to a measurement of fetal hemoglobin oxygen saturation according to, for example, one or more of the processes described herein.

A fetal hemoglobin probe 115, light source 105, and/or detector 160 may be of any appropriate size and, in some circumstances, may be sized to accommodate the size of the pregnant mammal and/or fetus using any appropriate sizing system (e.g., abdomen size and/or fetus age, etc.). Exemplary lengths for a fetal hemoglobin probe 115 include a length of 4 cm-40 cm and a width of 2 cm-10 cm. In some circumstances, the size and/or configuration of a fetal hemoglobin probe 115, or components thereof, may be responsive to skin pigmentation of the pregnant mammal and/or fetus. In some instances, the fetal hemoglobin probe 115 may be applied to the pregnant mammal's skin via tape or a strap that cooperates with a mechanism (e.g., snap, loop, etc.) (not shown).

System 100 includes a number of optional independent sensors/probes designed to monitor various aspects of maternal and/or fetal health and may be in contact with a pregnant mammal. These probes/sensors are a NIRS adult hemoglobin probe 125, a pulse oximetry probe 130, a Doppler and/or ultrasound probe 135, a uterine contraction measurement device 140, and an electrocardiogramavice 170. Not all embodiments of system 100 will include all of these components. ECG device 170 may be used to determine the pregnant mammal's and/or fetus' heart rate. In some embodiments, system 100 may further include an intrauterine pulse oximetry probe (not shown) that may be used to determine the fetus' heart rate. The Doppler and/or ultrasound probe 135 may be configured to be placed on the abdomen of the pregnant mammal and may be of a size and shape that approximates a silver U.S. dollar coin and may provide information regarding fetal position, orientation, and/or heart rate. Pulse oximetry probe 130 may be a conventional pulse oximetry probe placed on pregnant mammal's hand and/or finger to measure the pregnant mammal's hemoglobin oxygen saturation. NIRS adult hemoglobin probe 125 may be placed on, for example, the pregnant mammal's 2nd finger and may be configured to, for example, use near infrared spectroscopy to calculate the ratio of adult oxyhemoglobin to adult de-oxyhemoglobin. NIRS adult hemoglobin probe 125 may also be used to determine the pregnant mammal's heart rate.

Optionally, system 100 may include a uterine contraction measurement device 140 configured to measure the strength and/or timing of the pregnant mammal's uterine contractions. In some embodiments, uterine contractions will be measured by uterine contraction measurement device 140 as a function of pressure (measured in e.g., mmHg) over time. In some instances, the uterine contraction measurement device 140 is and/or includes a tocotransducer, which is an instrument that includes a pressure-sensing area that detects changes in the abdominal contour to measure uterine activity and, in this way, monitors frequency and duration of contractions.

In another embodiment, uterine contraction measurement device 140 may be configured to pass an electrical current through the pregnant mammal and measure changes in the electrical current as the uterus contracts. Additionally, or alternatively, uterine contractions may also be measured via near infrared spectroscopy using, for example, light received/detected by detector 160 because uterine contractions, which are muscle contractions, are oscillations of the uterine muscle between a contracted state and a relaxed state. Oxygen consumption of the uterine muscle during both of these stages is different and these differences may be detectable using NIRS.

Measurements and/or signals from NIRS adult hemoglobin probe 125, pulse oximetry probe 130, Doppler and/or ultrasound probe 135, and/or uterine contraction measurement device 140 may be communicated to receiver 145 for communication to computer 150 and display on display device 155 and, in some instances, may be considered secondary signals. As will be discussed below, measurements provided by NIRS adult hemoglobin probe 125, pulse oximetry probe 130, a Doppler and/or ultrasound probe 135, uterine contraction measurement device 140 may be used in conjunction with fetal hemoglobin probe 115 to isolate a fetal pulse signal and/or fetal heart rate from a maternal pulse signal and/or maternal heart rate. Receiver 145 may be configured to receive signals and/or data from one or more components of system 100 including, but not limited to, fetal hemoglobin probe 115, NIRS adult hemoglobin probe 125, pulse oximetry probe 130, Doppler and/or ultrasound probe 135, uterine contraction measurement device 140, and/or ECG device 170. Communication of receiver 145 with other components of system may be made using wired or wireless communication

In some instances, one or more of NIRS adult hemoglobin probe 125, pulse oximetry probe 130, a Doppler and/or ultrasound probe 135, uterine contraction measurement device 140, and/or ECG device 170 may include a dedicated display that provides the measurements to, for example, a user or medical treatment provider. It is important to note that not all of these probes may be used in every instance. For example, when the pregnant mammal is using fetal hemoglobin probe 115 in a setting outside of a hospital or treatment facility (e.g., at home or work) then, some of the probes (e.g., NIRS adult hemoglobin probe 125, pulse oximetry probe 130, a Doppler and/or ultrasound probe 135, uterine contraction measurement device 140) of system 100 may not be used.

In some instances, receiver 145 may be configured to process or pre-process received signals to, for example, make the signals compatible with computer 150 (e.g., convert an optical signal to an electrical signal), improve SNR, amplify a received signal, etc. In some instances, receiver 145 may be resident within and/or a component of computer 150. Also, receiver 145 is not limited to single receiver, as any number of appropriate receivers (e.g., 2, 3, 4, 5) may be used to receive signals from system 100 components and communicate them to computer 150. In some embodiments, computer 150 may amplify or otherwise condition the received reflected signal so as to, for example, improve the signal-to-noise ratio

Receiver 145 may communicate received, pre-processed, and/or processed signals to computer 150. Computer 150 may act to process the received signals, as discussed in greater detail below, and facilitate provision of the results to a display device 155. Exemplary computers 150 include desktop and laptop computers, servers, tablet computers, personal electronic devices, mobile devices (e.g., smart phones), and the like. Exemplary display devices 155 are computer monitors, tablet computer devices, and displays provided by one or more of the components of system 100. In some instances, display device 155 may be resident in receiver 145 and/or computer 150.

In some embodiments, a pregnant mammal may be electrically insulated from one or more components of system 100 by, for example, an electricity isolator 120. Exemplary electricity isolators 120 include transformers and non-conducting materials.

Fetal hemoglobin probe 115 may be used to direct NIR light into the abdomen of the pregnant mammal so as to reach the fetus and to detect light incident upon the fetus. The NIR light may be emitted by fetal hemoglobin probe 115 in, for example, a continuous and/or pulsed manner. This reflected light might then be processed in order to determine how much light, at various wavelengths, is reflected and/or absorbed by the fetal oxyhemoglobin and/or de-oxyhemoglobin so that a fetal hemoglobin oxygen saturation level may be determined. This processing will be discussed in greater detail below. In some embodiments, fetal hemoglobin probe 115 may be configured, partially or wholly, as a single-use, or disposable, probe that is affixed to the pregnant mammal's skin on, for example, the pregnant mammal's abdomen and, in some embodiments, in the supra-pubic (bikini) region.

Exemplary dimensions for fetal hemoglobin probe 115 include, but are not limited to, 2-16 inches in length and 0.5-8 inches in width. In some instances, fetal hemoglobin probe 115 may come in a variety of sizes so as to, for example, accommodate varying clinical needs, the size of the fetus, fetal position, the size of the pregnant mammal, and/or the size of the pregnant mammal's abdomen.

The fetal hemoglobin probe(s) 115 disclosed herein may include a housing 102 configured to house one or more components of fetal hemoglobin probe 115. Although the embodiments disclosed herein have all of the components of fetal hemoglobin probes 115 contained within a single housing 102, this is not necessarily the case as, for example, two or more components of a fetal hemoglobin probe 115 may be housed in separate housings 102. Housings 102 may be, for example, square, circular, or rectangular in shape and may be designed to be, in some instances, adjustable depending on, for example, a topology of the pregnant mammal's abdomen, a level of skin pigmentation for the pregnant mammal and/or her fetus, and so on.

In some embodiments, fetal hemoglobin probe 115 and/or housing 102 may be disposable and in other embodiments, fetal hemoglobin probe 115 (including and/or housing 102) may be configured for multiple uses (i.e., reusable). In some embodiments, (e.g., when fetal hemoglobin probe is configured to be disposable), may include an adhesive designed to be applied to the skin of the pregnant mammal's abdomen (e.g., glue, tape, etc.) configured to apply housing 102/fetal hemoglobin probe 115 directly to the skin of the pregnant mammal's abdomen and hold it in place there in a manner similar to a sticker. In some instances, the fetal hemoglobin probe 115 may be applied to the pregnant mammal's skin via tape or a strap that cooperates with a mechanism (e.g., snap, loop, etc.) (not shown) provided by the housing 102. In some circumstances, housing 102 may be attached/adjacent to the pregnant mammal's skin so that it does not move, and, in other instances, it may be allowed to move in order to, for example, attain better measurements/readings. In some cases, housing 102 and/or a portion thereof may not be adapted to be in contact with the pregnant mammal's abdomen.

In some embodiments, housing 102 and/or a portion thereof may cooperate with a reusable and/or disposable sleeve (not shown) that fits over fetal hemoglobin probe 115 so that fetal hemoglobin probe 115 may be placed within a housing 102 reusable and/or disposable sleeve so that it may be applied to the pregnant mammal's skin.

Fetal hemoglobin probe 115 may be adapted to direct, or shine, light of one or more wavelengths into the abdomen of a pregnant mammal and receive a signal corresponding to a reflection of a portion of that light from the pregnant mammal's tissue and fluid as well as the tissue and fluids of the fetus.

Optionally, fetal hemoglobin probe 115 may include one or more mechanisms that enable the emitted light to be directed in a particular direction. Such mechanisms include, but are not limited to, wedges or adhesive material, that may be transparent or substantially transparent. For example, a fetal hemoglobin probe 115 may include a wedge positioned on one side that operates to direct the light in a particular direction relative to the surface of the pregnant mammal's skin and/or position a detector or transceiver to receive an optimized amount of reflected light.

In some embodiments, a fetal hemoglobin probe 115 may be adapted to be worn by a pregnant mammal for an extended period of time (e.g., days, weeks, etc.) that is not necessarily coincident with the labor and delivery process in order to, for example, monitor the health of a fetus. In some embodiments, one or more components of fetal hemoglobin probe 115 may be positioned outside the fetal hemoglobin probe 115 and may be optically connected thereto via, for example, one or more fiber optic or Ethernet cable(s).

A fetal hemoglobin probe 115 may be of any appropriate size and, in some circumstances, may be sized so as to accommodate the size of the pregnant mammal using any appropriate sizing system (e.g., waist size and/or small, medium, large, etc.). Exemplary lengths for a fetal hemoglobin probe 115 include a length of 4 cm-40 cm and a width of 2 cm-10 cm. In some circumstances, the size and/or configuration of a fetal hemoglobin probe 115, or components thereof, may be responsive to skin pigmentation of the pregnant mammal and/or fetus.

It will be understood that although the components of fetal hemoglobin probe 115 are described herein as being included in a single probe, that is not necessarily so as the components of fetal hemoglobin probe 115 may be present in two or more different objects/devices applied to a pregnant mammal. In some instances, more than one fetal hemoglobin probe 115 may be used so as to, for example, improve accuracy of the fetal oxygen saturation measurement. For example, a first fetal hemoglobin probe 115 (or a component thereof) may be placed on a left side of a pregnant mammal's abdomen and a second fetal hemoglobin probe 115 (or a component thereof) may be placed on a right side of the pregnant mammal's abdomen.

The trans-abdominal fetal oximetry processes disclosed herein may be executed, at least in part, with a fetal hemoglobin probe that includes a plurality of detectors. In general, light may be directed into the abdomen of a pregnant mammal by one or more light sources fetal hemoglobin probe resident within a housing. Some of this light may be reflected by the pregnant mammal and/or the fetus and may then be received by one of a plurality of detectors resident within the fetal hemoglobin probe housing. Power may be supplied to a fetal hemoglobin probe via, for example, a battery or an external power supply.

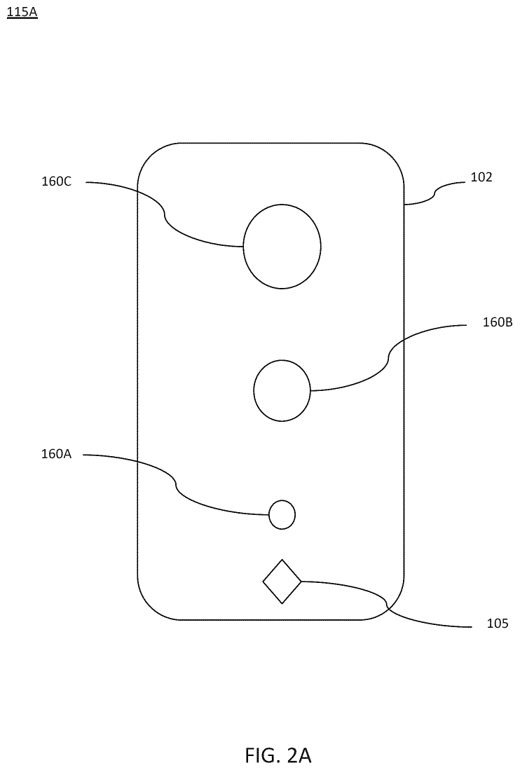

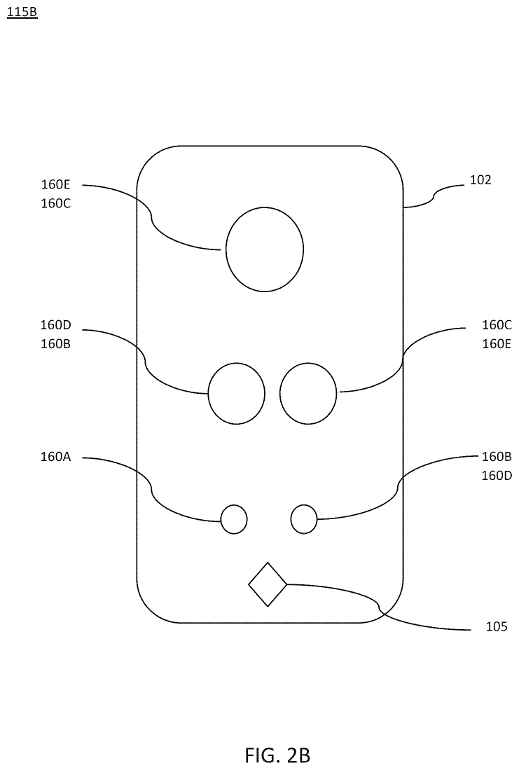

Example of a fetal hemoglobin probe are provided by FIGS. 2A and 2B, which provide an illustration of an exemplary fetal hemoglobin probes 115A and 115B, respectively. Fetal hemoglobin probe 115A includes one light source 105, a first detector 160A, a second detector 160B, and a third detector 160C positioned within a housing 102. In fetal hemoglobin probe 115A, first detector 160A is positioned closest (e.g., 0.5-3 cm) to light source 105, third detector 160C is positioned furthest away (e.g., 3-10 cm) from light source 105, and second detector 160B is positioned between first and third detectors 105A and 105C, respectively. Exemplary distances between light source 105 and detector 160B are 0.5-8 cm.

Fetal hemoglobin probe 115B includes light source 105, first detector 160A, second detector 160B, third detector 160C, a fourth detector 160D, and a fifth detector 160E positioned within a housing 102. In fetal hemoglobin probe 115B, first detector 160A and fourth detector 160D are positioned in a row that approximately aligns in the center of a horizontal axis between detectors 160A and 160D. An approximate distance between light source 105 and (as measured along a Y-axis bisecting a center of light source 105) and a center of the horizontal axis between first and fourth detectors 160A and 160D is 0.5-3 cm. Second detector 160B and fifth detector 160E are positioned in a row that approximately aligns in the center of a horizontal axis between detectors 160B and 160D. In most instances, the first and fourth detectors 160A and 160D and/or second and fifth detectors 160B and 160E will be the same distance from light source 105 (as shown in FIG. 2B) but this need not always be the case. For example, detector 160A may be 2 cm from light source 105 and detector 160B may be 4 cm from light source 105. An approximate distance between light source 105 and (as measured along a Y-axis bisecting a center of light source 105) and a center of the horizontal axis between second and fifth detectors 160B and 160E is 0.5-8 cm. Third detector 160C may be approximately 3-10 cm away from light source 105. In some instances, fetal hemoglobin probe 115 may include multiple light sources 105. In some embodiments, the number and/or configuration of the components of fetal hemoglobin probe 115A and/or 115B may be different than what is shown in FIGS. 2A and 2B. For example, fetal hemoglobin probe 115A and/or 115B may have multiple light sources 105, more detectors 160, and/or fewer detectors.

Detectors 160A-160E may be adapted to detect a light and/or optical signal emanating from the abdomen of a pregnant mammal. The detected optical signal may correspond to light projected by light source 105 and incident upon the pregnant mammal and/or the fetus. Detectors 160 and/or 160A-160E and convert this light signal into a digital and/or electronic signal (referred to herein as a “detected electronic signal”), which may be communicated to a receiver like receiver 145, a computer like computer 150, a processor like processor 604 and/or an on-board transceiver that may be capable of communicating the signal to the computer/processor.

The components of fetal hemoglobin probe 115 are arranged so that a first and second detector 160A and 160B, respectively, may be positioned approximately 1.5-4 cm (along the Y-axis) from the light source 105 and there may be a distance of approximately 1-4 cm (along the Y-axis) between them.

Due to their relatively close proximity to light source 105, the signals detected by first and/or fourth (when present) detector(s) 160A and 160D may detect a signal primarily generated by light reflected by the pregnant mammal, with little contribution from light incident upon the fetus, and these signals may be used to, for example, determine motion artifacts of the pregnant mammal, maternal photoplethysmogram information (e.g., photoplethysmogram variation), maternal heartbeat information, and so on. In embodiments where, for example, ICA is employed to isolate a fetal signal from a total signal, a signal from first detector 160A may be used to determine, for example, a first type of signal/information for the pregnant mammal (e.g., photoplethysmogram information) and fourth detector 160D may be used to determine a second type of signal/information for the pregnant mammal (e.g., motion artifact, maternal respiratory signal, or pregnant mammal heartbeat information). For purposes of the following discussion, the term respiration or respiratory is used to refer to the movement of a volume of gas into and out of the lungs, also known as ventilation.

In some circumstances, first and/or fourth detector(s) 160A and 160D may be smaller and/or relatively less sensitive (e.g., lower gain) than other detectors resident in housing 102. Even though first and/or fourth detector(s) 160A and 160D may be smaller and/or relatively less sensitive, it is expected that they will still detect a signal of adequate strength due, at least in part, on their respective relatively close proximity to the light source 105. Using detectors of smaller size/lower sensitivity for first and/or fourth detector(s) 160A and 160D may serve to, for example, reduce the cost of manufacturing fetal hemoglobin probe 102 and decrease the overall size of fetal hemoglobin probe 102, which may make wearing fetal hemoglobin probe 102 more comfortable for the pregnant mammal.

Because second and fifth detectors 160B and 160E are positioned further away from light source 105, a greater portion of a signal detected by these detectors (when compared to the signals detected by detectors 160A and 160D) may be incident upon the fetus. Stated differently, because the light incident upon the pregnant mammal's abdomen is expected to be more diffuse further away from the light source, a detector positioned further away from the light source may be expected to detect light incident upon a more diffuse area of the pregnant mammal's abdomen, including the fetus contained therein. Thus, it is likely that a greater proportion of the signals detected by second and fifth detectors 160B and 160E will be incident upon the fetus. This may result in detected electronic signal with a higher fetal/pregnant mammal ratio. The two signals detected by second and fifth detectors 160B and 160E may then be correlated with, for example, one another, a signal detected by first detector 160A, a signal detected by fourth detector 160D, and/or one or more secondary signals (e.g., fetal heartbeat) in order to, for example, amplify or otherwise strengthen the portion of the signal incident upon the fetus (fetal signal) and/or isolate the fetal signal from the total signal.

Third detector 160C may be of the same size and/or gain as second and/or fifth detectors 160C and/or 160E and, in other embodiments, may be larger in size and/or higher in gain so as to, for example, detect a signal of sufficient strength given its relative distance from light source 105.

A signal detected by fifth detector 160E may provide a signal with a higher fetal/pregnant mammal ratio when compared with the signals detected by first, second, fourth, and/or fifth detectors 160A, 160B, 160D, and/or 160E due, in part, to its proximity to light source 105.

In some embodiments, a detector 160 may be a sensitive camera adapted to capture small changes in fetal skin tone caused by changes in cardiovascular pressure as the fetus' heart beats. In these embodiments, fetal hemoglobin probe 115 may be in contact with the pregnant mammal's abdomen, or not, as this embodiment may be used to perform so-called contactless pulse oximetry. In these embodiments, light source(s) 105 of fetal hemoglobin probe 115 may be adapted to provide light (e.g., in the visible spectrum, near-infrared, etc.) directed toward the pregnant mammal's abdomen so that the detector 160 is able to receive light reflected by the pregnant mammal's abdomen and fetus. The reflected light captured by a detector 160A-160E in this embodiment may be communicated to computer 150 for processing so as to convert the images to a measurement of fetal hemoglobin oxygen saturation according to, for example, one or more of the processes described herein.

It will be appreciated that a fetal hemoglobin probe 115 may include any number of light sources and/or detectors. In some cases, more than one fetal hemoglobin probe 115 may be used. Additionally, or alternatively, a fetal hemoglobin probe 115 may be used in addition to an external light source 105 and/or detector 160.

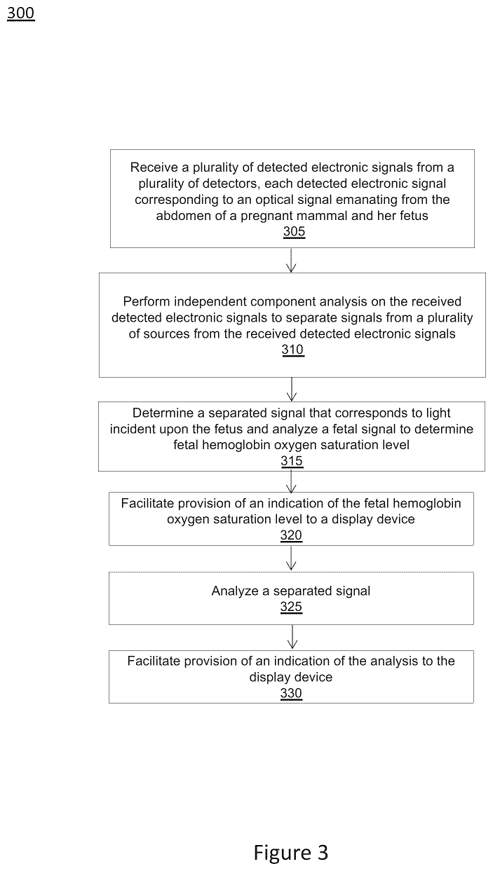

FIG. 3 provides a flowchart illustrating an exemplary process 300 for performing fetal oximetry and/or fetal pulse oximetry trans-abdominally and/or in-utero to determine fetal hemoglobin oxygen saturation level using independent component analysis. Process 300 may be performed by, for example, system 100 and/or a component thereof and/or a computer system like system 600 and/or a component thereof.

In step 305, a plurality of detected electronic signals may be received from a plurality (e.g., 3, 4, 5, 6, 7, 8, 9, etc.) of detectors, like detector 160, by a receiver such as receiver 145, and/or by a processor such as a processor 604. Each of the received detected electronic signals may correspond to an optical signal received by a detector included in the plurality of detectors, such as detectors 160A, 160B, 160C, 160D, or 160E that has been converted into a digital and/or electronic signal, which may be referred to herein as a “detected electronic signal.” The optical signal received by each detector may correspond to an optical signal projected into the abdomen of the pregnant mammal from one or more light sources, like light source 105, that emanates (e.g., reflection, backscattering, and/or transmission) from the abdomen of the pregnant mammal and her fetus. The received detected electronic signals may include light or photons that have been incident upon one or more layers of maternal and/or fetal tissue. In addition, the detected electronic signals may include portions, or signals, that are contributed by different sources including, but not limited to, maternal respiration, maternal photoplethysmogram variation, uterine tone changes, fetal photoplethysmogram, noise, motion artifacts, etc.

Often times, the light directed into the pregnant mammal's abdomen and the fetus will be of at least two separate wavelengths and/or frequencies (e.g., red, infrared, near-infrared, etc.) and the received detected electronic signals may correspond to light of these different wavelengths. For example, in one embodiment, there may be five detectors present in a fetal hemoglobin probe, such as exemplary fetal hemoglobin probe 115A, as shown in FIG. 2 and discussed above. Light projected into the pregnant mammal's abdomen by, for example, light source 105, may emanate from the pregnant mammal's abdomen and fetus, and may be detected by one or more of detectors 160A-160E. The respective detector 160A-160E may then convert the light they detect into a detected electronic signal and these detected electronic signals may be received by, for example, a receiver like receiver 145, a processor like processor 604, and/or a computer like computer 150.

In step 310, independent component analysis (ICA) may be executed to separate multiple signals that may be included within one or more of the detected electronic signals received in step 305. Each of the signals that are separated by the ICA from the plurality of detected electronic signals may be generated by a different source that may be associated with, for example, the pregnant mammal's body, her fetus, or noise. Exemplary sources of the signals that may be separated by ICA include, but are not limited to, maternal respiration, maternal photoplethysmogram variations, fetal photoplethysmogram variations, uterine tone changes, and motion artifacts. Often times, the ICA may be performed to generate a separated signal for maternal respiration, a separated signal for maternal photoplethysmogram variations, a separated signal for fetal photoplethysmogram variations, and a separated signal for noise. It will be appreciated that the ICA may generate separated signals from a variety of sources that may be different from/interchanged with those described above. In some instances, the ICA may generate separate signals proportionally to the number of detected electronic signals it received (i.e., three detected electronic signals yields three separated signals; four detected electronic signals yields four separated signals, etc.).

In some embodiments, execution of step 310 may include using blind source separation to separate out the signals contributed by the different sources. Additionally, or alternatively, execution of the ICA may be based on, or otherwise include, a maximum likelihood estimation (MLE). An objective of the ICA may be to isolate a portion of the received detected electronic signals that corresponds to light that has been incident upon the fetus and/or isolate a fetal photoplethysmogram signal. At times, a portion of the detected received signals that corresponds to the light that has been incident upon and/or incident upon the fetus may be referred to herein as a “fetal signal.”

Next, a separated signal associated with light that was incident upon the fetus (often times a fetal photoplethysmogram signal) may be analyzed to determine a fetal hemoglobin oxygen saturation level (step 315). In some embodiments, the fetal signal may correspond to the fetal photoplethysmogram signal. In some embodiments, execution of step 315 may include determining a ratio of a first wavelength of light (e.g., red light) included in the fetal signal and a second wavelength of light included in the fetal signal (e.g., near-infrared (NIR) light) and this ratio may be used to determine the fetal hemoglobin oxygen saturation level via known correlations between this ratio and the oxygen saturation of fetal hemoglobin via, for example, use of the Beer-Lambert Law and/or the Modified Beer-Lambert Law. Provision of the determined fetal hemoglobin oxygen saturation level to a user (e.g., doctor or nurse) may then be facilitated via, for example, communication of the fetal hemoglobin oxygen saturation level to a display device (e.g., display screen of a computer) like display device 155 (step 320). In some embodiments, step 320 may be performed by providing the user with a numerical value and/or graph showing the fetal hemoglobin oxygen saturation level and/or changes to fetal hemoglobin oxygen saturation level. Additionally, or alternatively, the fetal hemoglobin oxygen saturation level may be provided as a time weighted average taken over, for example, 30 seconds and/or 1, 2, 5, 10, 20, and/or 30 minutes.

Optionally, in some embodiments, one or more of the separated signals produced in step 310 may be analyzed (step 325) in order to, for example, monitor a source of the respective separated signal(s) under analysis. For example, if one of the signals separated in step 310 corresponds to a maternal respiratory signal, then this separated signal may be analyzed to determine one or more features of the pregnant mammal's breathing and/or respiratory cycle. Additionally, or alternatively, if one of the signals separated in step 310 corresponds to a uterine tone signal (which may indicate a muscular state of the uterus), then this separated signal may be analyzed to determine one or more features of the contractions and/or muscle tone of the pregnant mammal's uterus. Additionally, or alternatively, if one of the signals separated in step 310 corresponds to a maternal photoplethysmogram, then this separated signal may be analyzed to determine one or more features of the pregnant mammal's hemoglobin oxygen saturation. Then, results of this analysis may be provided to the user via, for example, communication of the results to a display device (step 330).

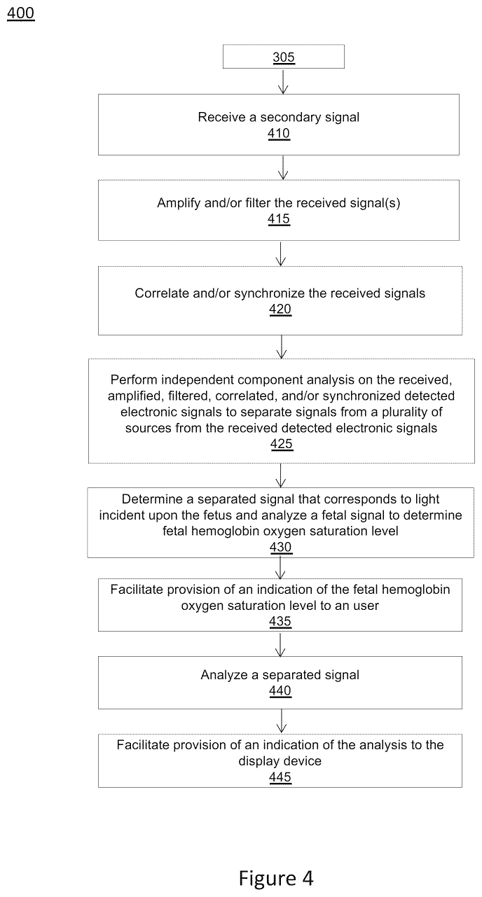

FIG. 4 provides a flowchart illustrating an exemplary process 400 for performing fetal oximetry and/or fetal pulse oximetry trans-abdominally and/or in-utero to determine fetal hemoglobin oxygen saturation level using independent component analysis. Process 400 may be performed by, for example, system 100 and/or a component thereof and/or a computer system like system 600 and/or a component thereof.

Initially, step 305 may be performed. Then, one or more secondary signal(s) may be received in step 410. In some instances, the secondary signals may be signals that are not detected optical signals or correlated to detected optical signals and, in other instances, the secondary signals may be derived from the detected optical signals. Exemplary secondary signals include, but are not limited to, a heartbeat signal for the pregnant mammal, as may be provided by an ECG device like ECG device 170, a heartbeat signal for the fetus, as may be provided by an ultrasound device Doppler/ultrasound probe 135, a pulse oximetry signal for the pregnant mammal as may be provided by a pulse oximetry probe like pulse oximetry probe 130, an indication of the pregnant mammal's hemoglobin oxygen saturation as may be provided by NIRS adult hemoglobin probe 125, and/or in indication of uterine tone and/or contractions as may be provided by uterine contraction measurement device.

In step 415, the signals received in step 305 and 410 may be amplified, filtered, or otherwise processed in order to, for example, reduce noise and/or amplify the signal, or portions thereof. In most instances, step 415 will be executed separately for each received signal. In some embodiments, execution of step 415 may include execution of known amplification and/or filtration techniques.

In some embodiments, execution of step 415 may include processing one or more of the signals received in step 305 with a secondary signal received in step 410. For example, if the secondary signal is a maternal heartrate signal (as may be received from, for example, ECG device 170), then the maternal heartrate signal may be used to determine which portions of the signals received in step 305 are contributed by the pregnant mammal. These portions may then be subtracted and/or divided from the signals received in step 305 to remove maternal contributions to the signals received in step 305. Additionally, or alternatively, if the secondary signal is a fetal heartrate signal, then the fetal heartrate signal may be used to determine which portions of the signals received in step 305 are contributed by the fetus because, for example, the oxygenation level of the fetus may fluctuate in a manner that corresponds to its pulse or heartrate. These portions of the signals received in step 305 may then be amplified by, for example, multiplying them by the fetal signal produced by subtracting portions of the maternal contributions from the signals received in step 305 and/or subtraction of portions of the signals received in step 305 that are not contributed by the fetus.

In other embodiments, the filtering of one or more of the received detected electronic signal(s) of step 415 may employ using one or more wavelet filters to filter the signal. Wavelet filters use a “mother wavelet” as the shape of the filter and, in order to improve the quality (e.g., signal-to-noise ratio) of a received signal, different mother wavelets (e.g., standard mother wavelets) may be used to optimize the signal being filtered. Additionally, or alternatively, a mother wavelet may be tailored to filter out portions of a received signal that may not correspond to the fetal signal, or what the fetal signal may be expected to be. An exemplary tailored, or specific, wavelet filter/mother wavelet may be derived from, for example, prototypical fetal signals that may be derived from, for example, fetal heartbeat signals (as provided by for example, Doppler/ultrasound probe 135), an arterial pressure sensor, or a fetal pulse oximetry signal. In some instances, this tailored, or specific, mother wavelet may be generated in real time (or near real time) as it is being used to filter one or more of the received detected electronic signals.

Additionally, or alternatively, filtering of one or more of the signals in step 415 may include evaluating the signal using undecimated and/or decimated multivariate empirical mode decomposition filter banks so as to, for example, create a filter tailored for a specific signal (e.g., the fetal signal, or a portion of the signal incident upon the pregnant mammal's breathing or uterine contractions).

Additionally, or alternatively, filtering and/or amplifying of one or more of the signals may include one or more of quantifying noise and other signal levels, evaluating fast Fourier transforms (FFTs), determining how harmonics and fetal signals overlap, removing motion artifacts (as may be caused by, for example, uterine contractions and/or maternal respiration), determining whether there is adequate optical signal amplitude, evaluating signals for sufficient signal amplitude, removal of portions of a signal that are below a quality (e.g., signal-to-noise ratio) threshold, and evaluating signals for sufficient signal capture length.

Additionally, or alternatively, filtering and/or amplifying of one or more of the signals may include one or more of creation of an ultrasound envelope corresponding to the arterial pressure pulse of the fetus, band pass filtering one or more of the signals to reduce portions of the respective signal received from non-fetal sources, detrending the received optical signals for breathing (of the pregnant mammal) and a DC signal generated transmission of light through tissue that does not correspond to arterial pulsatile blood flow, creating a blood pressure synchronous pulse signal using, for example, fetal heartbeat data and/or a fetal blood pressure data, calculating the amplitude of the correlation signal of two or more optical (e.g., light in the red, near-infrared, and/or infrared frequency/wavelength ranges) signals, normalizing an AC pulsatile signal to the DC level, determining a ratio between the two or more frequencies/wavelengths (e.g., a red light/infrared light ratio) of light included in a signal received in step 305, corresponds to light, and calibrating the determined ratio to a fetal blood oxygenation saturation level.

Optionally, in step 420, two or more of the received signals may be correlated to one another and/or synchronized in, for example, the time and/or frequency domains. The synchronization and/or correlation performed in step 420 may include gating a first of the two or more received signals so that it corresponds in time to a second of the two or more received signals. In this way events present in the received two or more received signals may align in time so that events of a first signal line up in time with the corresponding event in the second signal. In this way, when the first signal is used to understand the second signal (or vice versa), the first and second signals may be correlated in time.

In most instances, the correlation and/or synchronization of step 420 is performed by synchronizing one or more of the secondary signals received in step 410 with one or more of the signals received in step 305. For instance, execution of step 420 may involve synchronizing the one or more of the signals received in step 305 with a fetal heart beat signal and/or fetal blood pressure signal received in step 410. Additionally, or alternatively, execution of step 420 may involve performing a correlation function with one or more signals (individually or jointly) received in step 305 with a fetal heart beat signal and/or fetal blood pressure signal received in step 410.

Additionally, or alternatively, execution of step 420 may include correlating and/or synchronizing the detected optical signals received by the different detectors (e.g., detectors 160A-160E). At times, this step may not be necessary due to, for example, the speed of light and the relatively short distances between the light source(s) and the respective detectors.

In step 425, ICA may be performed on the received, amplified, filtered, correlated, and/or synchronized detected electronic signals to separate out signals contributed by different sources. Execution of step 425 may be similar to execution of step 310 with the exception that the input signal may be different. Instead of the input signal being the plurality of detected electronic signals received in step 305, the ICA of step 425 may be performed on amplified, filtered, correlated, and/or synchronized signals as may be generated by execution of step 415 and/or 420.

Next, the separated signals may be analyzed to determine a separated signal that corresponds to light incident upon the fetus (which may be referred to herein as a “fetal signal”) and the fetal signal may be analyzed to determine a fetal hemoglobin oxygen saturation level (step 430). Often, execution of step 430 may resemble execution of step 315.

Provision of the determined fetal hemoglobin oxygen saturation level to a user (e.g., doctor or nurse) may then be facilitated via, for example, communication of the fetal hemoglobin oxygen saturation level to a display device (e.g., display screen of a computer) like display device 155 (step 435). In some embodiments, step 435 may be performed by providing the user with a numerical value and/or graph showing the fetal hemoglobin oxygen saturation level and/or changes to fetal hemoglobin oxygen saturation level. Additionally, or alternatively, the fetal hemoglobin oxygen saturation level may be provided as a time weighted average taken over, for example, 30 seconds and/or 1, 2, 5, 10, 20, and/or 30 minutes.

Optionally, in some embodiments, one or more of the separated signals produced in step 425 may be analyzed (step 440) in order to, for example, monitor a source of the respective separated signal(s) under analysis and results of this analysis may be provided to the user via, for example, communication of the results to a display device (step 445). Execution of steps 440 and 445 may resemble execution of steps 325 and 330, respectively.

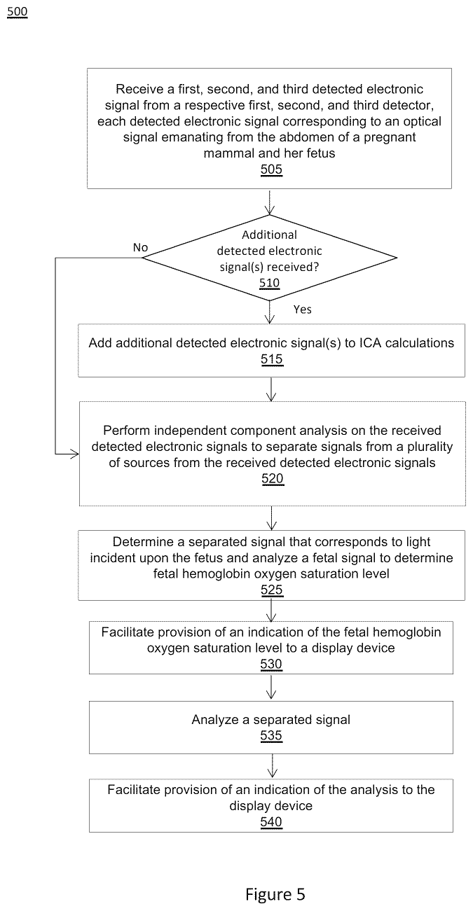

FIG. 5 provides a flowchart illustrating another exemplary process 500 for performing fetal oximetry and/or fetal pulse oximetry trans-abdominally and/or in-utero to determine fetal hemoglobin oxygen saturation level using independent component analysis. Process 500 may be performed by, for example, system 100 and/or a component thereof and/or a computer system like system 600 and/or a component thereof.

In step 505, a first detected electronic signal may be received from a first detector (e.g., detector 160A), a second electronic signal may be received from a second detector (e.g., detector 160B), and third detected electronic signal may be received a third detector (e.g., 160C) of fetal hemoglobin probe 115A or 115B. Each of the received detected electronic signals may correspond to an optical signal received by the respective first, second, and third detector that has been converted into a digital and/or electronic signal or detected electronic signal. The optical signal received by each detector may correspond to an optical signal projected into the abdomen of the pregnant mammal from one or more light sources, like light source 105, that emanates (e.g., reflection, backscattering, and/or transmission) from the abdomen of the pregnant mammal and her fetus. The received detected electronic signals may include light or photons that have been incident upon one or more layers of maternal and/or fetal tissue. In addition, the detected electronic signals may include portions, or signals, that are contributed by different sources including, but not limited to, maternal respiration, maternal photoplethysmogram variation, uterine tone changes, fetal photoplethysmogram, noise, motion artifacts, etc.

Often times, the light directed into the pregnant mammal's abdomen and the fetus will be of at least two separate wavelengths and/or frequencies (e.g., red, infrared, near-infrared, etc.) and the received detected electronic signals may correspond to light of these different wavelengths. For example, light projected into the pregnant mammal's abdomen by, for example, light source 105, may emanate from the pregnant mammal's abdomen and fetus, and may be detected by one or more of detectors 160A-160C. The respective detector 160A-160C may then convert the light they detect into a detected electronic signal and these detected electronic signals may be received by, for example, a receiver like receiver 145 and/or processor inside a computer like computer 150.

In step 510, it may be determined if additional (e.g., fourth, fifth, sixth, etc.) detected electronic signal(s) are received from additional detectors and, if so, the additional (e.g., fourth, fifth, sixth, etc.) detected electronic signal(s) may be added to the ICA calculations performed in step 520 (step 515). If not, process 500 proceeds to step 520.

In step 520, ICA may be executed to separate multiple signals that may be included within one or more of the detected electronic signals received in step 505. Each of the signals that are separated by the ICA from the plurality of detected electronic signals may be generated by a different source that may be associated with, for example, the pregnant mammal's body, her fetus, or noise. Exemplary sources of the signals that may be separated by ICA include, but are not limited to, maternal respiration, maternal photoplethysmogram variations, fetal photoplethysmogram variations, uterine tone changes, and motion artifacts. Often times, the ICA may be performed to generate a separated signal for maternal respiration, a separated signal for maternal photoplethysmogram variations, a separated signal for fetal photoplethysmogram variations, and a separated signal for noise. When only three detected electronic signals are received in step 505, the ICA may generate three separated signals. When additional detected electronic signals are received in step 510, the ICA may generate an additional separated signal for each additional detected electronic signal received.

In an embodiment where three detected electronic signals are received, the ICA may generate a first separated signal for maternal photoplethysmogram variations (source is maternal photoplethysmogram variations), a second separated signal for noise (source is noise), and a third separated signal for fetal photoplethysmogram variations (source is maternal photoplethysmogram variations). Alternatively, in some embodiments, the first or second separated signal may be for maternal respiration (source is maternal respiration) or uterine tone (source is maternal respiration). In embodiments where more than three detected electronic signals are received, a fourth separated signal may be for maternal respiration (source is maternal respiration) or uterine tone (source is changes in uterine tone). It will be appreciated that the ICA may generate separated signals from a variety of sources that may be different from/interchanged with those described above. In some instances, the ICA may generate separate signals proportionally to the number of detected electronic signals it received (i.e., three detected electronic signals yields three separated signals; four detected electronic signals yields four separated signals, etc.)

In some embodiments, execution of step 520 may include using blind source separation to separate out the signals contributed by the different sources. Additionally, or alternatively, execution of the ICA may be based on, or otherwise include, a maximum likelihood estimation (MLE). An objective of the ICA may be to isolate a portion of the received detected electronic signals that corresponds to light that has been incident upon the fetus and/or generate a separated fetal photoplethysmogram signal. At times, a portion of the detected received signals that corresponds to the light that has been incident upon and/or incident upon the fetus may be referred to herein as a “fetal signal.”

Next, a separated signal associated with light that was incident upon the fetus (often times a fetal photoplethysmogram signal) may be analyzed to determine a fetal hemoglobin oxygen saturation level (step 525). In some embodiments, execution of step 525 may include determining a ratio of a first wavelength of light (e.g., red light) included in the fetal signal and a second wavelength of light included in the fetal signal (e.g., near-infrared (NIR) light) and this ratio may be used to determine the fetal hemoglobin oxygen saturation level via known correlations between this ratio and the oxygen saturation of fetal hemoglobin via, for example, use of the Beer-Lambert Law and/or the Modified Beer-Lambert Law. Provision of the determined fetal hemoglobin oxygen saturation level to a user (e.g., doctor or nurse) may then be facilitated via, for example, communication of the fetal hemoglobin oxygen saturation level to a display device (e.g., display screen of a computer) like display device 155 (step 530). In some embodiments, step 530 may be performed by providing the user with a numerical value and/or graph showing the fetal hemoglobin oxygen saturation level and/or changes to fetal hemoglobin oxygen saturation level. Additionally, or alternatively, the fetal hemoglobin oxygen saturation level may be provided as a time weighted average taken over, for example, 30 seconds and/or 1, 2, 5, 10, 20, and/or 30 minutes.

Optionally, in some embodiments, one or more of the separated signals produced in step 520 may be analyzed (step 535) in order to, for example, monitor a source of the respective separated signal(s) under analysis. For example, if one of the signals separated in step 520 corresponds to a maternal respiratory signal, then this separated signal may be analyzed to determine one or more features of the pregnant mammal's breathing and/or respiratory cycle. Additionally, or alternatively, if one of the signals separated in step 520 corresponds to a uterine tone signal (which may indicate a muscular state of the uterus), then this separated signal may be analyzed to determine one or more features of the contractions and/or muscle tone of the pregnant mammal's uterus. Additionally, or alternatively, if one of the signals separated in step 520 corresponds to a maternal photoplethysmogram, then this separated signal may be analyzed to determine one or more features of the pregnant mammal's hemoglobin oxygen saturation. Then, results of this analysis may be provided to the user via, for example, communication of the results to a display device (step 540).

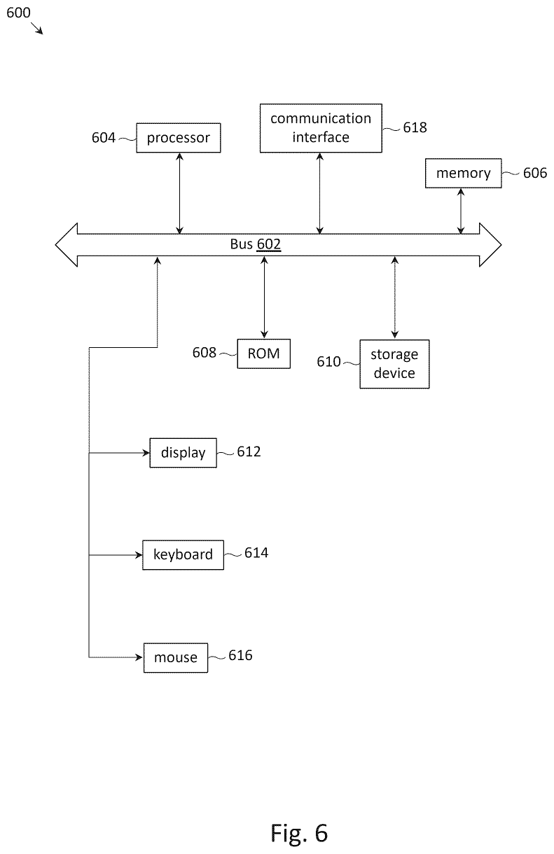

FIG. 6 provide an example of a processor-based system 600 that may store and/or execute instructions for the processes described herein. Processor-based system 600 may be representative of, for example, computing device 150. Note, not all of the various processor-based systems which may be employed in accordance with embodiments of the present invention have all of the features of system 600. For example, certain processor-based systems may not include a display inasmuch as the display function may be provided by a client computer communicatively coupled to the processor-based system or a display function may be unnecessary. Such details are not critical to the present invention.

System 600 includes a bus 602 or other communication mechanism for communicating information, and a processor 604 coupled with the bus 602 for processing information. System 600 also includes a main memory 606, such as a random access memory (RAM) or other dynamic storage device, coupled to the bus 602 for storing information and instructions to be executed by processor 604. Main memory 606 also may be used for storing temporary variables or other intermediate information during execution of instructions to be executed by processor 604. System 600 further includes a read only memory (ROM) 608 or other static storage device coupled to the bus 602 for storing static information and instructions for the processor 604. A storage device 610, which may be one or more of a floppy disk, a flexible disk, a hard disk, flash memory-based storage medium, magnetic tape or other magnetic storage medium, a compact disk (CD)-ROM, a digital versatile disk (DVD)-ROM, or other optical storage medium, or any other storage medium from which processor 604 can read, is provided and coupled to the bus 602 for storing information and instructions (e.g., operating systems, applications programs and the like).

System 600 may be coupled via the bus 602 to a display 612, such as a flat panel display, for displaying information to a user. An input device 614, such as a keyboard including alphanumeric and other keys, may be coupled to the bus 602 for communicating information and command selections to the processor 604. Another type of user input device is cursor control device 616, such as a mouse, a trackball, or cursor direction keys for communicating direction information and command selections to processor 604 and for controlling cursor movement on the display 612. Other user interface devices, such as microphones, speakers, etc. are not shown in detail but may be involved with the receipt of user input and/or presentation of output.

The processes referred to herein may be implemented by processor 604 executing appropriate sequences of processor-readable instructions stored in main memory 606. Such instructions may be read into main memory 606 from another processor-readable medium, such as storage device 610, and execution of the sequences of instructions contained in the main memory 606 causes the processor 604 to perform the associated actions. In alternative embodiments, hard-wired circuitry or firmware-controlled processing units (e.g., field programmable gate arrays) may be used in place of or in combination with processor 604 and its associated computer software instructions to implement the invention. The processor-readable instructions may be rendered in any computer language.

System 600 may also include a communication interface 618 coupled to the bus 602. Communication interface 618 may provide a two-way data communication channel with a computer network, which provides connectivity to the plasma processing systems discussed above. For example, communication interface 618 may be a local area network (LAN) card to provide a data communication connection to a compatible LAN, which itself is communicatively coupled to other computer systems. The precise details of such communication paths are not critical to the present invention. What is important is that system 600 can send and receive messages and data through the communication interface 618 and in that way communicate with other controllers, etc.

Hence, systems, devices, and methods for determining fetal oxygen level have been herein disclosed. In some embodiments, use of the systems, devices, and methods described herein may be particularly useful during the labor and delivery of the fetus (e.g., during the first and/or second stage of labor).

We claim:

1. A method comprising:
receiving, by a processor, a plurality of detected electronic signals, each of the plurality of detected electronic signals being received from a separate detector communicatively coupled to the processor and corresponding to a detected optical signal emanating from a pregnant mammal's abdomen and a fetus contained therein, wherein each detected optical signal has been converted, by the respective detector, into one of the plurality of the detected electronic signals;
performing, by the processor, independent component analysis on the plurality of detected electronic signals to separate signals within the detected electronic signals that are contributed by different sources, each of the separated signals corresponding to a different source;
determining, by the processor, a separated signal that corresponds to light incident upon the fetus;
analyzing, by the processor, the separated signal that corresponds to light incident upon the fetus to determine a fetal hemoglobin oxygen saturation level of the fetus; and
facilitating, by the processor, provision of an indication of the fetal hemoglobin oxygen saturation level to a user.

2. The method of claim 1, wherein the separated signal that corresponds to light incident upon the fetus is a fetal photoplethysmogram signal.

3. The method of claim 1, wherein the source of a separated signal is at least one of a maternal photoplethysmogram, a fetal photoplethysmogram, a maternal respiratory signal, a uterine tone signal, and a noise signal.

4. The method of claim 1, further comprising:
analyzing, by the processor, a separated signal that corresponds to light incident upon the pregnant mammal; and
facilitating, by the processor, provision of an indication of the analysis results to the user.

5. The method of claim 4, wherein the separated signal that corresponds to light incident upon the pregnant mammal is at least one of a maternal photoplethysmogram signal, a maternal respiratory signal, and a uterine tone signal.

6. The method of claim 1, further comprising:
filtering, by the processor, the received plurality of detected electronic signals prior to performance of the independent component analysis.

7. The method of claim 1, further comprising:
receiving, by the processor, a secondary signal; and
filtering, by the processor, the received plurality of detected electronic signals using the received secondary signal prior to performance of the independent component analysis.

8. The method of claim 7, further comprising:
synchronizing, by the processor, the secondary signal and the received plurality of detected electronic signals prior to filtering the received plurality of detected electronic signals.

9. The method of claim 1, wherein the optical signal includes at least two different wavelengths of light.

10. A method comprising:
receiving, by a processor, a plurality of detected electronic signals, each of the plurality of detected electronic signals being received from a separate detector communicatively coupled to the processor and corresponding to a detected optical signal emanating from a pregnant mammal's abdomen and a fetus contained therein, wherein each detected optical signal has been converted, by the respective detector, into one of the plurality of the detected electronic signals;
receiving, by the processor, a secondary signal;
filtering, by the processor, the received plurality of detected electronic signals using the received secondary signal;
performing, by the processor, independent component analysis on the plurality of filtered detected electronic signals to separate signals within the detected electronic signals that are contributed by different sources, each of the separated signals corresponding to a different source;
determining, by the processor, a separated signal that corresponds to light incident upon the fetus;
analyzing, by the processor, the separated signal that corresponds to light incident upon the fetus to determine a fetal hemoglobin oxygen saturation level of the fetus; and
facilitating, by the processor, provision of an indication of the fetal hemoglobin oxygen saturation level to a user.

11. The method of claim 10, wherein the separated signal that corresponds to light incident upon the fetus is a fetal photoplethysmogram signal.

12. The method of claim 10, wherein the source of a separated signal is at least one of a maternal photoplethysmogram, a fetal photoplethysmogram, a maternal respiratory signal, a uterine tone signal, and a noise signal.

13. The method of claim 10, further comprising:
analyzing, by the processor, a separated signal that corresponds to light incident upon the pregnant mammal; and
facilitating, by the processor, provision of an indication of the analysis results to the user.

14. The method of claim 13, wherein the separated signal that corresponds to light incident upon the pregnant mammal is at least one of a maternal photoplethysmogram signal, a maternal respiratory signal, and a uterine tone signal.

15. The method of claim 10, further comprising:
amplifying, by the processor, the received plurality of detected electronic signals prior to performance of the independent component analysis.

16. The method of claim 15, further comprising:
synchronizing, by the processor, the secondary signal and the received plurality of detected electronic signals prior to filtering the received plurality of detected electronic signals.

17. A method comprising:
receiving, by a processor, a first detected electronic signal from a first detector, a second detected electronic signal from a second detector, and a third detected electronic signal from a third detector, each of the first, second, and third detectors being communicatively coupled to the processor and each of the first, second, and third detected electronic signals corresponding to a detected optical signal emanating from a pregnant mammal's abdomen and a fetus contained that is detected by the respective first, second, and third detectors and converted into the respective first, second, and third detected electronic signals;
performing, by the processor, independent component analysis on the first, second, and third detected electronic signals to generate a first separated signal, a second separated signal, and a third separated signal, the first separated signal, second separated signal, and third separated signal being contributed to the first, second, and third detected electronic signals by a first source, a second source, and a third source, respectively, wherein the third separated signal corresponds to a fetal photoplethysmogram signal;
analyzing, by the processor, the third separated signal to determine a hemoglobin oxygen saturation level of the fetus; and
facilitating, by the processor, provision of an indication of the hemoglobin oxygen saturation level of the fetus to a user.

18. The method of claim 17, wherein the first source of the first separated signal is at least one of motion artifacts of the pregnant mammal, respiration of the pregnant mammal, photoplethysmogram variations of the pregnant mammal, uterine tone of the pregnant mammal, and noise.

19. The method of claim 17, wherein the second source of the second separated signal is at least one of motion artifacts of the pregnant mammal, respiration of the pregnant mammal, photoplethysmogram variations of the pregnant mammal, uterine tone of the pregnant mammal, and noise.

20. The method of claim 17, further comprising:
receiving, by the processor, a fourth detected electronic signal from a fourth detector communicatively coupled to the processor prior to performance of the independent component analysis, the fourth detected electronic signal corresponding to a detected optical signal emanating from the pregnant mammal's abdomen and fetus contained that is detected by the fourth detector and converted into the fourth detected electronic signal, wherein the independent component analysis is performed on the first, second, third, and fourth detected electronic signals to generate the first separated signal, the second separated signal, the third separated signal, and a fourth separated signal, the fourth separated signal being contributed by a fourth source.
